SEQUENCE LISTING (10 sequences)

SEQ ID NO: 1 - DNA, 163 bp, Homo sapiens
tgttttcctttacttactacacctcagatatatttcttcatgaagacctcacagtaaaaataggtgattttggtctagctacagtgaaatctcgatggagtgggtcccatcagtttgaacagttgtctggatccattttgtggatggtaagaattgaggctat

SEQ ID NO: 2 - DNA, 53 bp, Artificial Sequence
  Primer
tacggtagcagagacttggtctnnnnnnnntgatctatctgtgaaggttttca

SEQ ID NO: 3 - DNA, 22 bp, Artificial Sequence
  Overhang adaptor
tacggtagcagagacttggtct

SEQ ID NO: 4 - DNA, 28 bp, Artificial Sequence
  Primer
atagcctcaattcttaccatccacaaaa

SEQ ID NO: 5 - DNA, 67 bp, Artificial Sequence
  Primer
cgtatcgcctccctcgcgccatcagacgagtgcgttgttttcctttacttactacacctcagatata

SEQ ID NO: 6 - DNA, 25 bp, Artificial Sequence
  Adaptor
cgtatcgcctccctcgcgccatcag

SEQ ID NO: 7 - DNA, 10 bp, Artificial Sequence
  Barcode
acgagtgcgt

SEQ ID NO: 8 - DNA, 32 bp, Artificial Sequence
  Primer
tgttttcctttacttactacacctcagatata

SEQ ID NO: 9 - DNA, 63 bp, Artificial Sequence
  Primer
ctatgcgccttgccagcccgctcagacgagtgcgtatagcctcaattcttaccatccacaaaa

SEQ ID NO: 10 - DNA, 25 bp, Artificial Sequence
  Adaptor
ctatgcgccttgccagcccgctcag

(19) United States
(12) Patent Application Publication
(10) Pub. No.: US 2017/0016056 A1
(43) Pub. Date: Jan. 19, 2017

(54) ACCURATE DETECTION OF RARE GENETIC VARIANTS IN NEXT GENERATION SEQUENCING

(71) Applicant: GE HEALTHCARE BIO-SCIENCES CORP., Marlborough, MA (US)

(72) Inventors: Xin-Xing Tan, Houston, TX (US); Kenneth Bradford Thomas, Houston, TX (US); Lee Thomas Szkotnicki, Houston, TX (US); John Richard Nelson, Niskayuna, NY (US)

(21) Appl. No.: 15/124,286

(22) PCT Filed: Mar. 18, 2015
(86) PCT No.: PCT/US2015/021279

§ 371 Date: Sep. 7, 2016

Related U.S. Application Data
(60) Provisional application No. 61/971,792, filed on Mar. 28, 2014.

(51) Int. Cl. C12Q 1/68; C12P 19/34

(52) U.S. Cl. CPC C12Q 1/6855; C12P 19/34

(57) ABSTRACT

The invention relates to a method for analyzing a target nucleic acid fragment, comprising generating a first strand using one strand of the target as a template by primer extension, using a first oligonucleotide primer which comprises, from 5′ to 3′, an overhang adaptor region, a primer ID region and a target specific sequence region complementary to one end of the target fragment; optionally removing non-incorporated primers; amplifying the target from the generated first strand to produce an amplification product; and detecting the amplification product. Also disclosed are unique primers useful for such target analysis methods.

### FIELD OF THE INVENTION

The invention relates to a method for analyzing a target nucleic acid fragment. More specifically, the invention relates to the use of an oligonucleotide primer which contains a primer ID sequence for the analysis of a target nucleotide sequence. The invention further discloses oligonucleotide primers suitable for such use.

### BACKGROUND OF THE INVENTION

Next Generation Sequencing (NGS) technologies offer great opportunity to determine the occurrence and frequency of nucleotide mutations at the genomics level that contribute to certain phenotypes, e.g., cancer development, viral drug resistance, etc. However, the relatively high background error rates (from both sequencing technologies and the proceeding PCR amplifications in the procedure) confound the accurate detection of the true genetic variation. This is especially true when the variation is a SNP present at extremely low frequency in a highly heterogeneous sample population.

U.S. patent application US 2013/0310264 A1 provides a method for deep sequencing, by the analysis of random mixtures of non-overlapping genomic fragments. For RNA virus sequencing, the use of Primer ID was recently discussed. Jabara, C, et al., PNAS, 2011, v108, 20166-20171. See also WO2013/0130512.

### BRIEF SUMMARY OF THE INVENTION

The embodiments of the invention enable accurate detection of sequence variants with extremely low frequency in a nucleic acid sample. Therefore, it achieves identification of false positives and false negatives of any variants discovered, thus significantly increases the accuracy and sensitivity of variant detection.

Thus in one embodiment, the invention relates to a method for analyzing a target nucleic acid fragment. The method comprises
(a) generating a first strand using one strand of e target as a template by primer extension, using a first oligonucleotide primer which comprises, from 5′ to 3′, an overhang adaptor region, a primer ID region and a target specific sequence region complementary to one end of the target fragment;
(b) optionally removing non-incorporated primers;
(c) amplifying the target from the generated first strand to produce an amplification product; and
(d) detecting the amplification product.

In certain embodiments, the method further comprising, before the amplifying step,
(1) generating a second strand using the generated first strand as a template by primer extension, using a second oligonucleotide primer which comprises, from 5′ to 3′, a second overhang adaptor region, a second primer ID region and a target specific sequence region complementary to the other end of the target fragment; and
(2) optionally removing non-incorporated primers.

In certain embodiments, primer extension is achieved in the presence of a high-fidelity DNA polymerase.

In certain embodiments, the amplifying step is achieved by the polymerase chain reaction (PCR).

In another embodiment, the invention relates to a set of oligonucleotide primers, comprising
(1) a first oligonucleotide primer which comprises, from 5′ to 3′, an overhang adaptor region, a primer ID region and a target specific sequence region complementary to one end of a target fragment; and
second and third oligonucleotide primers as PCR primers,
   (i) the second comprising, from 5′ to 3′, a region complementary to a first sequencing primer, an optional barcode region and a region complementary to the overhang adapter region of the first primer; and
   (ii) the third comprising, from 5′ to 3′, a region complementary to a second sequencing primer, a second optional barcode region and a region complementary to the other end of the target fragment.

In another embodiment, the invention relates to a set of oligonucleotide primers, comprising
1) a first oligonucleotide primer which comprises, from 5′ to 3′, an overhang adaptor region, a primer ID region and a target specific sequence region complementary to one end of a target fragment; and
2) a second oligonucleotide primer which comprises, from 5′ to 3′, a second overhang adaptor region, a second primer ID region and a target specific sequence region complementary to the other end of the target fragment.

### BRIEF DESCRIPTION OF THE DRAWINGS

FIG. 1 shows a schematic for amplifying a target nucleic acid according to an embodiment of the invention.

FIG. 2 shows expected result of a BioAnalyzer QC of the amplicons

### DETAILED DESCRIPTION OF THE INVENTION

### Definitions

The singular forms “a” “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques.

The term “barcode” or “barcode region” as used here refers to a short polynucleotide region, such as 2-10 nucleotides, e.g., 2, 3, 4, 5, 6, 7, 8, 9, or 10 nucleotides. The barcode may represent an analysis date, time or location; a clinical trial; a collection date, time or location; a patient number; a sample number; a species; a subspecies; a subtype; a therapeutic regimen; or a tissue type.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be analyzed or amplified. See, M. Kanehisa Nucleic Acids Res. 12:203 (1984), incorporated herein by reference.

The term “consensus sequence” as used herein refers a sequence formed from two or more sequences containing an identical Primer ID.

High fidelity DNA polymerase: The fidelity of a DNA polymerase is the result of accurate replication of a desired template. Specifically, this involves multiple steps, including the ability to read a template strand, select the appropriate nucleoside triphosphate and insert the correct nucleotide at the 3′ primer terminus, such that Watson-Crick base pairing is maintained. In addition to effective discrimination of correct versus incorrect nucleotide incorporation, some DNA polymerases possess a 3′→5′ exonuclease activity. This activity, known as “proofreading”, is used to excise incorrectly incorporated mononucleotides that are then replaced with the correct nucleotide. High fidelity DNA polymerases are those that either have a low misincorporation rates or proofreading activity or both to give faithful replication of the target DNA of interest. Some example high fidelity DNA polymerases are T7 DNA polymerase, T4 DNA polymerase, phi29 DNA polymerase, Pfu DNA polymerase, DNA polymerase I and Klenow fragment of DNA polymerase I.

The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) that may be isolated from natural sources, recombinantly produced or artificially synthesized and mimetics thereof. A further example of a polynucleotide of the present invention may include non natural analogs that may increase specificity of hybridization, for example, peptide nucleic acid (PNA) linkages and Locked Nucleic Acid (LNA) linkages. The LNA linkages are conformationally restricted nucleotide analogs that bind to complementary target with a higher melting temperature and greater mismatch discrimination. Other modifications that may be included in probes include: 2′OMe, 2′OAllyl, 2′O-propargyl, 2′O- 2′O-alkyl, 2′ fluoro, 2′ arabino, 2′ xylo, 2′ fluoro arabino, phosphorothioate, phosphorodithioate, phosphoroamidates, 2′Amino, 5-alkyl-substituted pyrimidine, 5-halo-substituted pyrimidine, alkyl-substituted purine, halo-substituted purine, bicyclic nucleotides, 2′MOE, LNA-like molecules and derivatives thereof. The invention also encompasses situations in which there is a nontraditional base pairing such as Hoogsteen base pairing which has been identified in certain tPvNA molecules and postulated to exist in a triple helix. “Polynucleotide” and “oligonucleotide” are used interchangeably in this application.

The term “primer” or “oligonucleotide primer” as used herein refers to a double-stranded, single-stranded, or partially single-stranded oligonucleotide. In some embodiments, primers are capable of acting as a point of initiation for template-directed nucleic acid synthesis under suitable conditions for example, buffer and temperature, in the presence of four different nucleoside triphosphates and an agent for polymerization, such as, for example, DNA polymerase. Primers can be comprised of DNA or RNA or other nucleotide analogs. The length of the primer, in any given case, depends on, for example, the intended use of the primer, and generally ranges from 15 to 100 nucleotides. Short primer molecules generally require cooler temperatures to form sufficiently stable hybrid complexes with the template. A primer needs not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “primer ID” or “primer ID region” as used herein refers to a degenerate string of nucleotides introduced into a primer during the oligonucleotide synthesis reaction. As primers are synthesized de novo, a population of primers will contain unique combinations at that degenerate block. For example, a Primer ID containing a block of 8 degenerate bases will have 65,536 (4⁸) unique combinations. For example, a first Primer ID may be 5′GCATCTTC3′ and a second may be 5′CAAGTAAC3′. Each has a unique identity that can be determined by determining the identity and order of the bases in the Primer ID.

Next generation high-throughput sequencing protocols require a large amount of starting genomic material. Amplification such as PCR is typically a necessary first step in sequencing, as templates are limiting. During PCR, the polymerase will introduce errors into the amplification product. These errors will be reported by the high resolution of next generation sequencing platforms. A Primer ID allows for tracking of individual genomic fragments through the PCR and sequencing protocol and direct error correction. Without a Primer ID, artifactual errors have to be removed from biological diversity through statistical means, which is not always possible.

Embodiments of the invention allow for more accurate detection of nucleic acid fragments, such as by DNA sequencing, which decreases the read depth required in order to obtain highly accurate consensus DNA sequence. The methods also enable accurate detection of true variants presents in a target samples, especially for variants with extremely low percentage in a highly heterogeneous population, by directly removing error, identifying/filtering false positives in the discovered variants. In addition the methods may increase detection sensitivity of the next generation sequencing technologies by identifying false negatives.

Embodiments of the invention are especially suited for analyzing a sample where a target nucleic acid fragment is from an individual suffering from cancer. Embodiments of the invention are also suited for analyzing a sample where a target nucleic acid fragment is from cell-free, circulating nucleic acid. Rare variant sequences from such samples are readily detected by methods according to embodiments of the invention. Rare variant sequences refer to those with low frequency, e.g. 5% or lower.

In certain embodiments, the target nucleic acid fragment is a double stranded nucleic acid fragment. In certain embodiments, the double stranded nucleic acid fragment is a double stranded DNA fragment. In other embodiments, the target nucleic acid fragment is a single stranded nucleic acid fragment. In certain embodiments, the single stranded nucleic acid fragment is a single stranded DNA fragment.

In one aspect, the invention relates to a method for analyzing a target nucleic acid fragment. The method comprises
a) generating a first strand using one strand of the target as a template by primer extension, using a first oligonucleotide primer which comprises, from 5′ to 3′, an overhang adaptor region, a primer ID region and a target specific sequence region complementary to one end of the target fragment;
b) optionally removing non-incorporated primers;
c) amplifying the target from the generated first strand to produce an amplification product; and
d) detecting the amplification product.

The first strand synthesis uses a specially designed oligonucleotide primer. The primer comprises, from 5′ to 3′, an overhang adaptor region, a primer ID region and a target specific sequence region complementary to one end of the target fragment. The primers each includes a primer ID region which may be used after target amplification and subsequent detection step (e.g., sequencing) to determine which target sequences came from common starting template molecules. By using this method, any artifactual changes introduced into the nucleic acid product will become obvious, as each target is typically analyzed (e.g., sequenced) many times in one next-generation DNA sequencing run. By comparing the sequencing results from one primer ID, any differences in the sequence must be attributed to error, whether this be an amplification error or a sequencing error or any other artificial introduction of mistake into the DNA sequence.

In certain embodiments, the primer ID region comprises a degenerate sequence. In certain other embodiments, the primer ID region comprises 5-100 nucleotides. In still other embodiments, the primer ID region comprises 5-50 nucleotides. In a preferred embodiment, the primer ID region comprises at least 8 nucleotides. In some embodiments, the primer ID region comprises a predetermined sequence.

Toward the 3′ end of the primer, a target specific sequence region is included which is complementary to one end of the target fragment. This sequence is capable of annealing to the target fragment such that a DNA polymerase synthesizes the first strand by primer extension using the target as a template.

Toward the 5′ end of the primer, an overhang adaptor region is included which serves as the priming side for a primer for subsequent amplification of the synthesized first strand.

In certain embodiments, the primer extension reaction for first strand generation is performed in the presence of a high-fidelity DNA polymerase. Utilization of a high fidelity DNA polymerase instead of a typical DNA polymerase such as Taq polymerase to generate the first strand will decrease the rate of errors in this most important step by at least 10 times, such as 50 times, or 100 times. In certain preferred embodiments, the high-fidelity DNA polymerase(s) is/are selected from a T7 DNA polymerase, T4 DNA polymerase, phi29 DNA polymerase, Pfu DNA polymerase, DNA polymerase I and Klenow fragment of DNA polymerase I.

After the first strand is synthesized, an optional step may be introduced to remove the non-incorporated primers. The presence of such primers does not affect the final analysis of the target, however it may reduce amplification efficiency. Because the synthesized first strand and the primers differ in length (i.e., size), removal of the un-extended primers may be achieved by a size-based separation, such as a size-exclusion membrane. Excess primers may also be removed by nuclease digestion, as the primers are single stranded while the first strand synthesis products are double stranded.

In certain embodiments, to further increase accuracy of detection of the target, the method of analyzing a target nucleic acid fragment may comprise, before the amplifying step,
1) generating a second strand using the generated first strand as a template by primer extension, using a second oligonucleotide primer which comprises, from 5′ to 3′, a second overhang adaptor region, a second primer ID region and a target specific sequence region complementary to the other end of the target fragment; and
2) optionally removing non-incorporated primers.
   This second strand is generated using a primer with similar features to the first primer, under similar conditions as described in detail above.

In some embodiments, each step is performed under conditions where excess, unextended primers from the previous step will not hybridize to target, for instance at higher temperature.

After the generation of the first strand or the first and the second strand according to certain embodiments of the invention, an amplification step is employed to produce an amplification product.

In some embodiments, the amplification step comprises a non-PCR-based method. In some embodiments, the non-PCR-based method comprises multiple displacement amplification (MDA). In some embodiments, the non-PCR-based method comprises transcription-mediated amplification (TMA). In some embodiments, the non-PCR-based method comprises nucleic acid sequence-based amplification (NASBA). In some embodiments, the non-PCR-based method comprises strand displacement amplification (SDA). In some embodiments, the non-PCR-based method comprises real-time SDA. some embodiments, the non-PCR-based method comprises rolling circle amplification. In some embodiments, the non-PCR-based method comprises circle-to-circle amplification. In some embodiments the non-PCR method comprises helicase-dependent amplification (HDA). In some embodiments the non-PCR method comprises rolling circle amplification (RCA). There are many amplification methods known that can be used, and potentially new methods of amplification that could be used. This list in no way is limiting the methods that one skilled in the art may devise to amplify the product.

In some embodiments, the amplification step comprises a PCR-based method. In some embodiments, the PCR-based method comprises PCR. In some embodiments, the PCR-based method comprises quantitative PCR. In some embodiments, the PCR-based method comprises emulsion PCR. In some embodiments, the PCR-based method comprises droplet PCR. In some embodiments, the PCR-based method comprises hot start PCR. In some embodiments, the PCR-based method comprises in situ PCR. In some embodiments, the PCR-based method comprises inverse PCR. In some embodiments, the PCR-based method comprises multiplex PCR. In some embodiments, the PCR-based method comprises Variable Number of Tandem Repeats (VNTR) PCR. In some embodiments, the PCR-based method comprises asymmetric PCR. In some embodiments, the PCR-based method comprises long PCR. In some embodiments, the PCR-based method comprises nested PCR. In some embodiments, the PCR-based method comprises hemi-nested PCR. In some embodiments, the PCR-based method comprises touchdown PCR. In some embodiments, the PCR-based method comprises assembly PCR. In some embodiments, the PCR-based method comprises colony PCR.

In certain embodiments, when the synthesized first strand is used as a template for PCR, the PCR is performed with a pair of oligonucleotide primers, of which
(i) a first PCR primer comprising, from 5′ to 3′, an optional region complementary to a first sequencing primer, an optional barcode region and a region complementary to the overhang adapter region of the first primer; and
(ii) a second PCR primer comprising, from 5′ to 3′, an optional region complementary to a second sequencing primer, a second optional barcode region and a region complementary to the other end of the target fragment.

In certain embodiments, when the synthesized first and second strand are used as templates for PCR, the PCR is performed with a pair of oligonucleotide primers, of which
(i) a first PCR primer comprising, from 5′ to 3′, an optional region complementary to a first sequencing primer, an optional barcode region and a region complementary to the overhang adapter region of the first primer; and
(ii) a second PCR primer comprising, from 5′ to 3′, an optional region complementary to a second sequencing primer, a second optional barcode region and a region complementary to the second overhang adapter region of the second primer.

The presence on the first and second PCR primer of an optional region complementary to a sequencing primer enables the subsequent analysis of the amplified product by sequencing.

The presence on the first and/or second PCR primer of an optional barcode assigns a unique ID to each individual sample. Thus multiple samples can be pooled together for subsequent analysis of DNA sample from different source material.

In some embodiments, detecting the amplification product comprises sequencing the amplification product. Sequencing of the amplification product may occur by a variety of methods, including, but not limited to the Maxam-Gilbert sequencing method, the Sanger dideoxy sequencing method, dye-terminator sequencing method, pyrosequencing, multiple-primer DNA sequencing, shotgun sequencing, and primer walking. In some embodiments, sequencing comprises pyrosequencing. In some embodiments the sequencing comprises a next-generation DNA sequencing method. Sequencing primers may be designed such that it includes a 3′ region complementary to the optional region of the PCR primer which is complementary to the sequencing primer.

In some embodiments, detecting the amplification product further comprises counting a number of different Primer Ds associated with the amplification product, wherein the number of different Primer IDs associated with the amplification product reflects the number of templates sampled. In some embodiments, the method further comprises forming a consensus sequence for amplification product comprising the same Primer ID.

The method may further comprise detecting one or more genetic variants based on the detection of the amplification product. For example, genetic variants may be detected by sequencing the amplification product. Sequences with the same Primer ID can be grouped together to form a Primer ID family. A genetic variant can be detected when at least 50% of the amplification product in the Primer ID family contains the same nucleotide sequence variation. When less than 50% of the nucleic acid molecules in the Primer ID family contain the same nucleotide sequence variation, then the nucleotide sequence variation can be due to sequencing and/or amplification error. In some embodiments, detecting the genetic variants comprises determining the prevalence of mutations. In some embodiments, detecting the genetic variants comprises forming a consensus sequence for the amplification product comprising the same Primer ID.

In some embodiments, detecting genetic variants comprises counting a number of different amplification products. In some embodiments, the genetic variant comprises a polymorphism. In some embodiments, the polymorphism comprises a single nucleotide polymorphism. In some instances, the polymorphism occurs at a frequency of less than 0.5%. In some instances, the polymorphism occurs at a frequency of less than 1%. In some instances, the polymorphism occurs at a frequency of less than 2%. In some instances, the polymorphism occurs at a frequency of less than 5%. In some instances, the polymorphism occurs at a frequency of greater than 1%. In some instances, the polymorphism occurs at a frequency of greater than 5%. In some instances, the polymorphism occurs at a frequency of greater than 10%. In some instances, the polymorphism occurs at a frequency of greater than 20%. In some instances, the polymorphism occurs at a frequency of greater than 30%. In some embodiments, the genetic variant comprises a mutation. In some embodiments, the genetic variant comprises a deletion. In some embodiments, the genetic variant comprises a insertion.

In some embodiments, detecting the amplification product comprises sequencing the amplification product by next generation sequencing technology. Suitable next generation sequencing technologies are widely available for use in connection with the methods described herein. Examples include the 454 Life Sciences platform (Roche, Branford, Conn.); Illumina's Genome Analyzer (Illumina, San Diego, Calif.), HiSeq and MiSeq; Ion Torrent PGM and Proton (Life Technologies) or DNA Sequencing by Ligation, SOLiD System (Applied Biosystems/Life Technologies. These systems allow the sequencing of many nucleic acid molecules isolated from a specimen at high orders of multiplexing in a parallel fashion (Dear, 2003, Brief Funct. Genomic Proteomic, 1(4), 397-416 and McCaughan and Dear, 2010, J. Pathol., 220, 297-306). Each of these platforms allows sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Amplified target nucleic acids may be immobilized to a solid support, hybridized with a sequencing primer, incubated with DNA polymerase, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphsulfate, fueling the luciferin reaction, which produces a chemiluminescent signal allowing sequence determination. Machines for pyrosequencing and methylation specific reagents are available from Qiagen, Inc. (Valencia, Calif.). See also Tost and Gut, 2007, Nat. Prot. 2 2265-2275. An example of a system that can be used by a person of ordinary skill based on pyrosequencing generally involves the following steps: ligating an adaptor nucleic acid to a target nucleic acid and hybridizing the nucleic acid to a bead; amplifying a nucleotide sequence in the target nucleic acid in an emulsion; sorting beads using a picoliter multiwell solid support; and sequencing amplified nucleotide sequences by pyrosequencing methodology (e.g., Nakano et al., 2003, J. Biotech. 102, 117-124). Such a system can be used to exponentially amplify amplification products generated by a process described herein, e.g., by ligating a heterologous nucleic acid to the first amplification product generated by a process described herein.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing or detection, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each other for energy transfer to occur successfully. Bailey et al. recently reported a highly sensitive (15 pg methylated DNA) method using quantum dots to detect methylation status using fluorescence resonance energy transfer (MS-qFRET) (Bailey et al. 2009, Genome Res. 19(8), 1455-1461, which is incorporated herein by reference in its entirety).

An example of a system that can be used based on single-molecule sequencing generally involves hybridizing a primer to a amplified target nucleic acid to generate a complex; associating the complex with a solid phase; iteratively extending the primer by a nucleotide tagged with a fluorescent molecule; and capturing an image of fluorescence resonance energy transfer signals after each iteration (e.g., Braslavsky et al, PNAS 100(7): 3960-3964 (2003); U.S. Pat. No. 7,297,518). Such a system can be used to directly sequence amplification products generated by processes described herein. In some embodiments the released linear amplification product can be hybridized to a primer that contains sequences complementary to immobilized capture sequences present on a solid support, a bead or glass slide for example. Hybridization of the primer-released linear amplification product complexes with the immobilized capture sequences, immobilizes released linear amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer-released linear amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

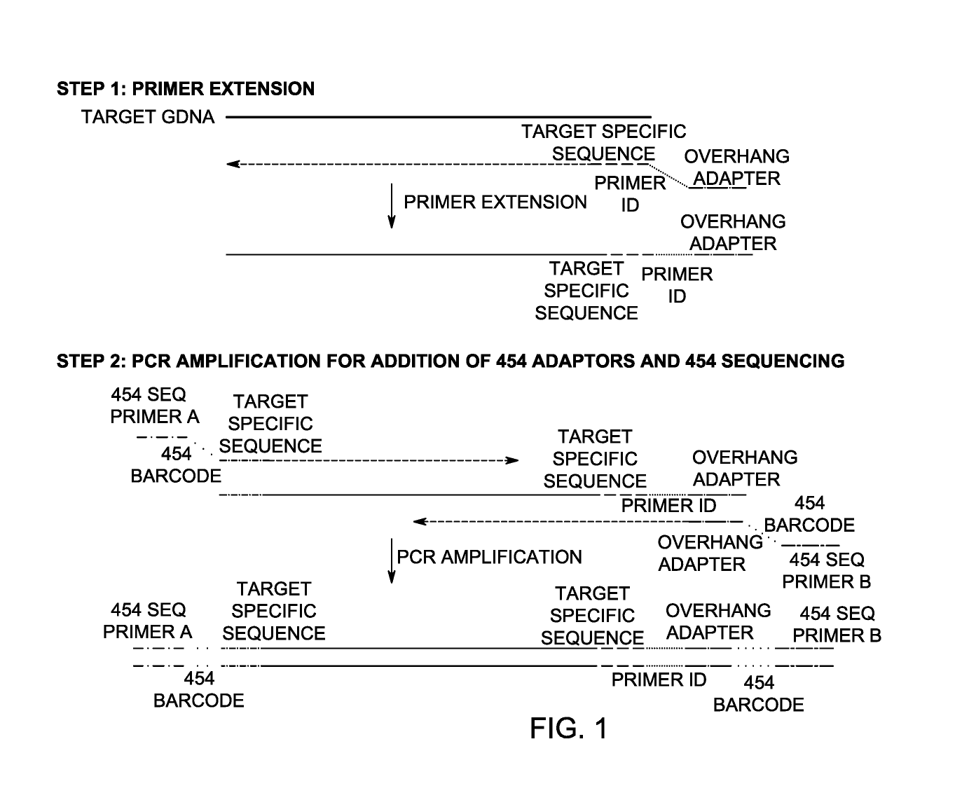

FIG. 1 illustrates a schematic for amplifying a target nucleic acid according to an embodiment of the invention.

A primer library was designed and synthesized including a target specific sequence, a primer ID, and an overhand adaptor. The Primer IDs are random sequence tags with eight or more bases in length. A similar primer may be designed for the other end of the target fragment. Although the Primer ID could be integrated in both forward and reverse primers, only one such primer is required (and shown) for the method to work. If the Primer ID is used in both the forward and reverse primers, two rounds of extension with a high fidelity DNA polymerase are needed to generate double-tagged products that can be amplified with generic PCR adapter primers. The overhand adaptor region provides a priming site for the subsequent downstream PCR with generic adapter primers.

In the primer extension step, the target specific sequence region of the primer is annealed to one strand of the target nucleic acid molecule in the samples and was extended using a high fidelity DNA polymerase to generate a single stranded “copy” of the original DNA molecule. The use of a high fidelity DNA polymerase ensures that the “copy” was made with 1×10⁻⁵-1×10⁻⁶ error rate. The generated “copy” now includes a unique sequence tag (Primer ID) and is used as template in the downstream PCR reaction, such that all the PCR products that come from the same original DNA molecule have the common Primer ID.

In the PCR amplification step, a special primer pair is used to amplify the single strand primer extension product. One of the PCR primers contains a 3′ sequence complementary to the overhang adapter region of the primer extension primer, as well as a 5′ region which is a complementary sequence to a sequencing primer. In this case the sequencing primer is the 454 sequencing primer B. The primer further includes a Barcode region. The other PCR primer contains a 3′ sequence identical to the other end of the single strand primer extension product (the target sequence), as well as a 5′ region which is a complementary sequence to the 454 sequencing primer A. This primer also includes a Barcode region. PCR amplification generates an amplified product for subsequence analysis, such as sequencing using a 454 sequencing machine.

Also disclosed are oligonucleotide primers useful for analyzing a template nucleic acid fragment. Thus, in one embodiment, the invention provides a set of oligonucleotide primers, comprising
(1) a first oligonucleotide primer which comprises, from 5′ to 3′, an overhang adaptor region, a primer ID region and a target specific sequence region complementary to one end of a target fragment; and
(2) second and third oligonucleotide primers as PCR primers,
   (i) the second comprising, from 5′ to 3′, a region complementary to a first sequencing primer, an optional barcode region and a region complementary to the overhang adapter region of the first primer; and
   (ii) the third comprising, from 5′ to 3′, a region complementary to a second sequencing primer, a second optional barcode region and a region complementary to the other end of the target fragment.

In another embodiment, the invention provides a set of oligonucleotide primers, comprising (1) a first oligonucleotide primer which comprises, from 5′ to 3′, an overhang adaptor region, a primer ID region and a target specific sequence region complementary to one end of a target fragment; and (2) a second oligonucleotide primer which comprises, from 5′ to 3′, a second overhang adaptor region, a second primer ID region and a target specific sequence region complementary to the other end of the target fragment. In certain embodiments, the set of primers further comprising a third and fourth oligonucleotide primers as PCR primers, the third primer comprising, from 5′ to 3′, a region complementary to a first sequencing primer, an optional barcode region and a region complementary to the overhang adapter region of the first primer; and the fourth primer comprising, from 5′ to 3′, a region complementary to a second sequencing primer, a second optional barcode region and a region complementary to the second overhang adapter region of the second primer.

### EXAMPLE

### Target Gene

Certain embodiments of the present invention are applied to detecting rare mutations (5% or lower frequency) for a region of interest V600 in the BRAF gene in human melanoma samples. Several mutations in this region have been implicated in malignant melanoma that is responsive to drug therapy.

Below is the DNA sequence of the target gene.

TGTTTTCCTTTACTTACTACACCTCAGATATATTTCTTCATGAAGACCTC
ACAGTAAAAATAGGTGATTTTGGTCTAGCTACAGTGAAATCTCGATGGAG
TGGGTCCCATCAGTTTGAACAGTTGTCTGGATCCATTTTGTGGATGGTAA
GAATTGAGGCTAT

### Primer Design

As described in FIG. 1 above, primers are designed and synthesized for primer extension (step 1) and PCR amplification (step 2):

### Primer for Primer Extension:

| Primer Name | Primer Sequence |
|---|---|
| BRAF_E | TACGGTAGCAGAGACTTGGTCTNNNNNNNNT |
| | GATCTATCTGTGAAGGTTTTCA |
| Primer Components | Sequence |
| Overhang Adaptor | TACGGTAGCAGAGACTTGGTCT |
| Primer ID | NNNNNNNN |
| Target-Specific | ATAGCCTCAATTCTTACCATCCACAAAA |

### Forward Primer for PCR:

| Primer Name | Primer Seq |
|---|---|
| Forward Primer | CGTATCGCCTCCCTCGCGCCATCAGACGAGTGC |
| | GTTGTTTTCCTTTACTTACTACACCTCAGATA |
| | TA |
| Primer | |
| Components | Sequence |
| 454 Adaptor | CGTATCGCCTCCCTCGCGCCATCAG |
| 454 Barcode | ACGAGTGCGT |
| Target-Specific | TGTTTTCCTTTACTTACTACACCTCAGATATA |

Reverse Primer for the Step 2

| Primer Name | Primer Seq |
|---|---|
| Reverse Primer | CTATGCGCCTTGCCAGCCCGCTCAGACGAGT |
| | GCGTATAGCCTCAATTCTTACCATCCACAAA |
| | A |
| Primer Components | Sequence |
| 454 Adaptor | CTATGCGCCTTGCCAGCCCGCTCAG |
| 454 Barcode | ACGAGTGCGT |
| Overhang Adaptor | TACGGTAGCAGAGACTTGGTCT |

Preparation of Sequencing-Ready Amplicon with Primer ID

1. Add the following items to a 96-well elate and mix well.

| | |
|---|---|
| gDNA (50 ng/ul) | 5 ul |
| Extension Primer (10 uM) | 5 ul |
| Oligo Hybridization Buffer | 40 ul |

2. Place the tube in a pre-heated block at 95° C. and incubate for 1 minute.

3. Set the temperature of the pre-heated block to 40° C. and continue incubating for 80 minutes.

4. After the 80 minute incubation, transfer the entire volume of sample onto the center of pre-washed wells of a filter plate (Millipore). The filter plate was pre-washed by adding 45 ul of wash buffer and centrifuging at 2,400 g at RT for 2 minute.

5. Centrifuge the filter plate at 2,400 g at RT for 2 minutes.

6. Wash the filter plate twice by adding 45 ul of wash buffer and centrifuging at 2,400 g for 2 minutes.

7. Make a master mix containing the following components and add it onto the center of the filter plate.

| | |
|---|---|
| phi29 DNA Polymerase | 5 ul |
| phi29 DNA Polymerase Reaction Buffer (10×) | 5 ul |
| dNTP (20 mM) | 5 ul |
| H2O | 35 ul |

8. Incubate the plate at 30° C. for 45 minutes

9. After the 45 minute incubation, centrifuge the filter plate at 2,400 g at RT for 2 minute and wash the plate twice as step 6.

10. Prepare master mix containing the following components and transfer it onto the center of the filter plate.

| | | |
|---|---|---|
| 10× PCR Buffer with MgCl₂ | 5 | ul |
| Forward Primer (10 uM) | 5 | ul |
| Reverse Primer (10 uM) | 5 | ul |
| dNTP (20 mM) | 5 | ul |
| AmpliTaq (Life Technologies) | 0.5 | ul |
| H₂O | 29.5 | ul |

11. Perform PCR using the following program on a thermal cycler:
95° C. for 3 minutes
25 cycles of: 95° C. for 30 s; 62° C. for 30 s; 72° C. for 60 s.
72° C. for 5 minutes
Hold at 10° C.

12. Transfer 1 ul of the PCR product to a single tube and add 45 ul of AMPure XP beads (Beckman Coulter) and vortex.

13. Incubate at RT without shaking for 10 minutes.

14. Place the tube on a magnetic stand for 2 minutes, and then remove the supernatants.

15. Wash the bead twice with 200 ul 80% ethanol.

16. Remove the tube from the magnetic stand and allow the beads to air-dry for 10 minutes.

17. Add 30 ul of TE buffer to the tube and vortex.

18. Incubate at RT without shaking for 2 minutes.

19. Place the tube on the magnetic stand for 2 minutes, and transfer 20 ul of supernatant to a new tube.

20. Take 1 ul for BioAnalyzer QC (Agilent) to determine the library size and 1 ul for PicoGreen QC (Invitrogen) to measure the library concentration.

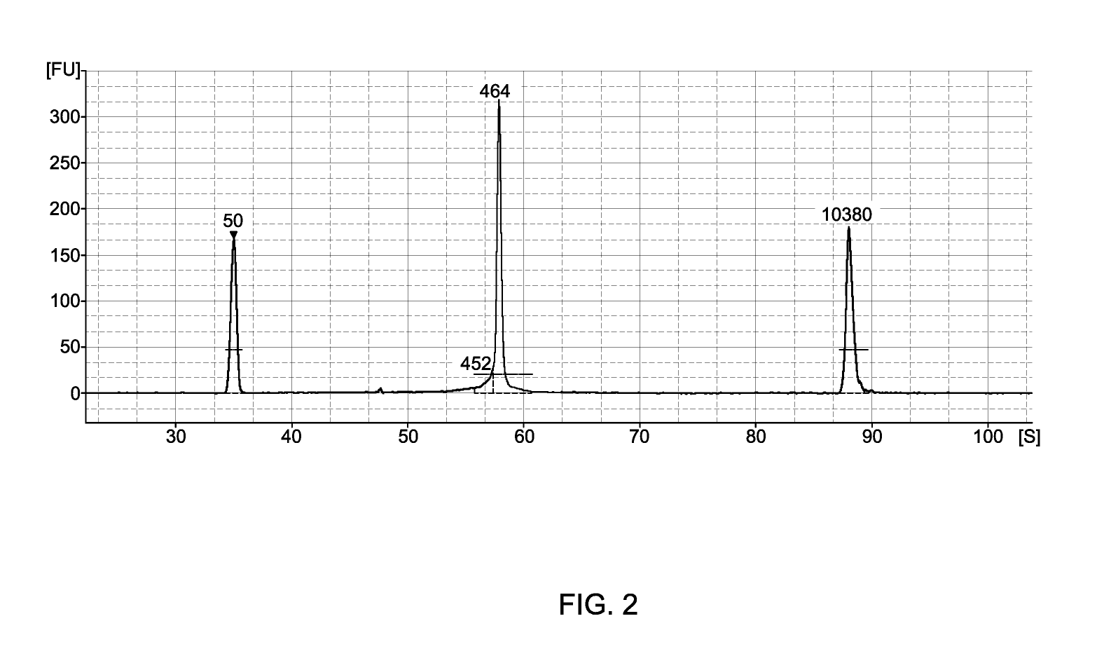

FIG. 2 shows the expected BioAnalyzer QC results

### 454 Sequencing of the Generated Amplicon

The generated amplicon library is used for 454 emPCR with the Roche/454 Amplicon emPCR kit following the manufacturer's instruction. The recovered beads are used for 454 sequencing following the manufacturer's manual as well.

### Data Analysis for Identification of Rare Variants

Read data, as from the 454 instrument, is extracted as base letter data that including any added barcode information. Data is segregated by barcode into ensembles of reads that have the same barcode by software that reads the random barcode and, while allowing no error in the barcode segregates the data into buffers. The data from each buffer is aligned and used to generate a consensus sequence based on simple majority at each position in the aligned sequence. Alternatively, quality score information can be used to weight the value of each base in its contribution to the consensus sequence. The consensus sequences are recorded as output and used in downstream methods such as variant calling. They may be treated as read sequences with no quality information, or quality information may be generated for them during consensus building.

Random sequences (i.e., primer IDs) do not uniquely label templates—this can be seen by examining labeling as a simple collision problem in probability. Given a primer ID length of L the number of possible primer IDs is B=4^{L}. If the total number of templates is N, the expected number of templates that will have the same primer ID is D=N(1−(1−1/B)^{N-1}). This assumes there is no bias for any of the primer IDs. For large numbers of templates, it becomes a significant possibility that an ensemble of reads identified by a primer ID contains amplification products from two or more templates. Analysis of samples is done by generation of consensus sequences from ensemble identified by the same primer ID, so this is a potential source of error. For primer ID lengths 8 through 12 the following Table shows the expected number of templates that share at least one primer ID expressed as a percentage (in one significant digit) of the total number of templates.

Total Number of Templates

| | 100 | 200 | 300 | 400 | 500 | 600 | 700 | 800 | 900 | 1000 | 10000 | 20000 |
|---|---|---|---|---|---|---|---|---|---|---|---|---|
| 8 | 0.2 | 0.3 | 0.5 | 0.6 | 0.8 | 0.9 | 1.1 | 1.2 | 1.4 | 1.5 | 14.2 | 26.3 |
| 9 | 0 | 0.1 | 0.1 | 0.2 | 0.2 | 0.2 | 0.3 | 0.3 | 0.3 | 0.4 | 3.7 | 7.3 |
| 10 | 0 | 0 | 0 | 0 | 0 | 0.1 | 0.1 | 0.1 | 0.1 | 0.1 | 0.9 | 1.9 |
| 11 | 0 | 0 | 0 | 0 | 0 | 0 | 0 | 0 | 0 | 0 | 0.2 | 0.5 |
| 12 | 0 | 0 | 0 | 0 | 0 | 0 | 0 | 0 | 0 | 0 | 0.1 | 0.1 |

Two strategies are apparent for minimizing the contribution of such collisions of primer IDs to error. Increasing primer ID length is one. The other is limiting the amount of template DNA. The latter method imposes lower limits on variant frequency that can be detected. For example, at 500× coverage (500 templates) the probability by binomial calculations that the variant appears at least 4 times with at least one read in each direction is 69% (these conditions are regarded as confirmatory for an apparent variant).

The utility of the random primer ID method is clear when it's considered what happens with an ensemble of 100 reads from a single template. A false variant call would require that an apparent variant appear in >50% of the reads off of the template in order for it to appear in the consensus of the ensemble. The probability that the 1% error rate of 454 sequencing could lead to a variant call at a particular position is less than 1×10⁻¹⁵ assuming there is no bias at that position. Thus, an apparent variant in such an ensemble does, at very high probability, represent a feature in the template that gave rise to it.

This written description uses examples to disclose the invention, including the preferred embodiments, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for analyzing a target nucleic acid fragment, comprising
a) generating a first strand using one strand of the target as a template by primer extension, using a first oligonucleotide primer which comprises, from 5′ to 3′, an overhang adaptor region, a primer ID region and a target specific sequence region complementary to one end of the target fragment;
b) optionally removing non-incorporated primers;
c) amplifying the target from the generated first strand to produce an amplification product; and
d) detecting the amplification product.

2. The method of claim 1, further comprising, before the amplifying step,
1) generating a second strand using the generated first strand as a template by primer extension, using a second oligonucleotide primer which comprises, from 5′ to 3′, a second overhang adaptor region, a second primer ID region and a target specific sequence region complementary to the other end of the target fragment; and
2) optionally removing non-incorporated primers.

3. The method of claim 1, wherein the target nucleic acid fragment is from an individual suffering from cancer.

4. The method of claim 1, wherein the target nucleic acid fragment is from cell-free, circulating nucleic acid.

5. The method of claim 1, wherein the generating step includes the use of a high-fidelity DNA polymerase.

6. The method of claim 5, wherein the high-fidelity DNA polymerase is selected from a proof-reading DNA polymerase, such as T7 DNA polymerase, T4 DNA polymerase, phi29 DNA polymerase, Pfu DNA polymerase, DNA polymerase I and Klenow fragment of DNA polymerase I.

7. The method of claim 1, wherein amplifying comprises a non-PCR-based method.

8. The method of claim 7, wherein the non-PCR-based method comprises multiple displacement amplification (MDA), nucleic acid sequence-based amplification (NASBA), helicase dependent amplification (HDA), rolling circle amplification (RCA) or strand displacement amplification (SDA).

9. The method of claim 1, wherein the amplifying step comprises a PCR-based method.

10. The method of claim 9, wherein the PCR-based method comprises PCR.

11. The method of claim 10, wherein the PCR is performed with a pair of oligonucleotide primers,
(i) a first PCR primer comprising, from 5′ to 3′, an optional region complementary to a first sequencing primer, an optional barcode region and a region complementary to the overhang adapter region of the first primer; and
(ii) a second PCR primer comprising, from 5′ to 3′, an optional region complementary to a second sequencing primer, a second optional barcode region and a region complementary to the other end of the target fragment.

12. The method of claim 2, wherein the amplifying step comprises a PCR-based method.

13. The method of claim 12, wherein the PCR-based method comprises PCR.

14. The method of claim 13, wherein the PCR is performed with a pair of oligonucleotide primers,
(i) a first PCR primer comprising, from 5′ to 3′, an optional region complementary to a first sequencing primer, an optional barcode region and a region complementary to the overhang adapter region of the first primer; and
(ii) a second PCR primer comprising, from 5′ to 3′, an optional region complementary to a second sequencing primer, a second optional barcode region and a region complementary to the second overhang adapter region of the second primer.

15. The method of claim 1, for detecting a rare variant sequence.

16. The method of claim 15, wherein detecting the amplification product comprises sequencing the amplification product.

17. The method of claim 16, further comprising forming a consensus sequence for each amplification product from the same Primer ID.

18. The method of claim 16, further comprising determining the prevalence of mutations.

19. The method of claim 15, wherein the rare sequence comprises a polymorphism

20. The method of claim 19, wherein the polymorphism comprises a single nucleotide polymorphism.

21. The method of claim 15, wherein the rare sequence comprises a mutation.

22. The method of claim 15, wherein the rare sequence comprises a deletion.

23. The method of claim 15, wherein the rare sequence comprises an insertion.

24. The method of claim 1, wherein the primer ID region comprises a degenerate sequence.

25. The method of claim 1, wherein the primer ID region comprises 5-100 nucleotides.

26. The method of claim 1, wherein the primer ID region comprises 5-50 nucleotides.

27. The method of claim 1, wherein the primer ID region comprises at least 8 nucleotides.

28. The method of claim 1, wherein the primer ID region comprises a predetermined sequence.

29. A set of oligonucleotide primers, comprising
1) a first oligonucleotide primer which comprises, from 5′ to 3′, an overhang adaptor region, a primer ID region and a target specific sequence region complementary to one end of a target fragment; and
2) second and third oligonucleotide primers as PCR primers,
a) the second comprising, from 5′ to 3′, a region complementary to a first sequencing primer, an optional barcode region and a region complementary to the overhang adapter region of the first primer; and
b) the third comprising, from 5′ to 3′, a region complementary to a second sequencing primer, a second optional barcode region and a region complementary to the other end of the target fragment.

30. A set of oligonucleotide primers, comprising
(1) a first oligonucleotide primer which comprises, from 5′ to 3′, an overhang adaptor region, a primer ID region and a target specific sequence region complementary to one end of a target fragment; and
(2) a second oligonucleotide primer which comprises, from 5′ to 3′, a second overhang adaptor region, a second primer ID region and a target specific sequence region complementary to the other end of the target fragment.

31. The set of oligonucleotide primers of claim 30, further comprising a third and fourth oligonucleotide primers as PCR primers,
(i) the third primer comprising, from 5′ to 3′, a region complementary to a first sequencing primer, an optional barcode region and a region complementary to the overhang adapter region of the first primer; and
(ii) the fourth primer comprising, from 5′ to 3′, a region complementary to a second sequencing primer, a second optional barcode region and a region complementary to the second overhang adapter region of the second primer.
